(19) United States
(12) Patent Application Publication
(10) Pub. No.: US 2006/0270683 A1
(43) Pub. Date: Nov. 30, 2006

(54) Polymorphs of aripiprazole

(76) Inventors: Braj Bhusha Lohray, Gujarat (IN); Vidya Bhushan Lohray, Gujarat (IN); Kaushik Babubhai Sata, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/554,366

(22) PCT Filed: Apr. 22, 2004
(86) PCT No.: PCT/IN04/00113

§ 371 Date: Feb. 6, 2006

(30) Foreign Application Priority Data
Apr. 25, 2003 (IN) 412/MUM/2003
Jun. 6, 2003 (IN) 583/MUM/2003

(51) Int. Cl. A61K 31/496; C07D 403/02

(52) U.S. Cl. 514/253.07; 544/363

(57) ABSTRACT

The present invention relates to new polymorphs of Aripiprazole, process for their preparation, pharmaceutical compositions containing them and their use.

### FIELD OF INVENTION

The present invention relates to novel polymorphs of Aripiprazole, process for preparing them, and pharmaceutical compositions containing the same and their use as a central nervous controlling agents specially in the treatment of mental disorders.

### BACKGROUND OF THE INVENTION

Aripiprazole, 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro-2(1H)-quinolinone, is an atypical antipsychotic agent useful as a central nervous controlling agent, preferably, for the treatment of schizophrenia. The molecular structure of Aripiprazole is represented by formula (1).

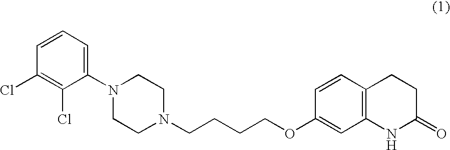

Schizophrenia is one of the most serious mental disorders. It may include withdrawal from reality, disorders of thought processes, abnormal behaviour and a gross inability to communicate with other people. It is the most common type of psychosis and affects up to one person in every hundred. Onset of schizophrenia typically occurs between the age of 16 and 25. It is commonly characterized by delusions, hallucinations and extensive withdrawal from others.

Aripiprazole is marketed under the brand name ‘Abilify’ by Bristol Myers Squibb and is indicated for the treatment of Alzheimer's dementia, antipsychotic disorders and bipolar disorders.

Aripiprazole as carbostyril derivatives have been disclosed in U.S. Pat. No. 4,734,416 and U.S. Pat. No. 5,006,528 for the treatment of schizophrenia.

US Patent Application No. 2002193438 discloses a pharmaceutical composition suitable for oral administration comprising aripiprazole.

EP Patent No. 1145711 discloses granules for the production of flash-melt pharmaceutical oral dosage forms of aripiprazole.

The present invention discloses new forms of Aripiprazole. We hereby describe three such new polymorphs as well as process for preparing the same.

A further aspect of the present invention is to disclose a pharmaceutical composition and dosage form containing the novel forms of Aripiprazole

### OBJECTS OF THE INVENTION

It is an object of the present invention to provide-new polymorphic forms of Aripiprazole.

A further object of the present invention is to provide processes for the preparation of the new polymorphic forms of Aripiprazole.

A still further object of the present invention is to provide pharmaceutical compositions and dosage forms comprising of the novel forms of Aripiprazole described herein, and their use for the treatment of psychological disorders.

Yet, another object of the present invention is to provide pharmaceutical composition containing a mixture of one or more polymorphs of Aripiprazole selected from forms II to IV mixtures thereof.

### SUMMARY OF INVENTION

The present invention provides a novel polymorph of Aripiprazole characterized by data selected from the group comprising of DSC thermogram with an endothermic peal; in the range of 133-137° C., X-ray diffraction pattern with peaks at about 5.820, 8.730, 11.640, 15.800, 16.310, 17.710, 18.610, 21.220, 22.090, 23.390, 24.950, 26.410, 30.970 and 34.190±0.2 degrees two-theta.

1 aa preferred embdodiment, the novel polymorph of Aripiprazole is characterized by data selected from the group comprising of DSC thermogram with an endothermic peak in the range of 122-124° C., X-ray diffraction pattern with peaks at about 8.730, 10.310, 12.170, 15.600, 16.700, 17.490, 19.30, 19.850, 20.440, 21.490, 22.180, 23.370, 24.510, 25.450, 26.940, 27.910, 28.407, 34.980, 137.080±0.2 degrees two-theta.

In another preferred embodiment, the novel polymorph of Aripiprazole is characterized by data selected from the group comprising of DSC thermogram with an endothermic peak in the range of 146-149° C., X-ray diffraction pattern with peaks at about 5.510, 10.250, 11.580, 12.760, 13.890, 15.540, 16.130, 17.240, 18.170, 18.540, 19.080, 20.740, 21.550, 22.240, 23.660, 24.740, 25.140, 25.740, 26.480, 27.010, 28.320, 29.450, 31.000, 32.600, 33.600, 35.590, 36.980, 38.840±0.2 degrees two-theta.

The present invention also provides a process for the preparation of the novel polymorph of Aripiprazole comprising,
a) contacting/dissolving crude Aripiprazole with suitable solvents selected from the group consisting of isopropanol, isopropyl acetate, methanol or mixtures thereof, at elevated temperature followed by cooling.
b) removing the solvent.

In a preferred embodiment, the process for the preparation of the polymorph of Aripiprazole comprises
a) contacting/dissolving crude Aripiprazole with suitable solvents selected from the group consisting of isobutyl acetate, ethanol or mixtures thereof, at elevated temperature followed by cooling.
b) removing the solvent.

In another preferred embodiment, the process for the preparation of the polymorph of Aripiprazole comprises:
a) contacting/Dissolving crude Aripiprazole with suitable solvents selected from the group consisting of acetone, t-butanol or mixtures thereof, at elevated temperature followed by cooling.
b) removing the solvent.

### DESCRIPTION OF FIGURES

FIG. 1: DSC of Form II of Aripiprazole having DSC endotherm peak at 135° C.

FIG. 2: XRD pattern of Form II of Aripiprazole

FIG. 3: DSC of Form III of Aripiprazole having DSC endotherm peak at 124° C.

FIG. 4: ED pattern of Form III of Aripiprazole

FIG. 5: DSC of Form IV of Aripiprazole-having DSC endotherm peak at 147.6° C.

FIG. 6: XRD pattern of Form IV of Aripiprazole

### DETAILED DESCRIPTION

As used herein the term “crude form” refers to crystals of a compound that have not been washed and/or recrystallised to remove impurities that may be present.

As used herein the term “crystalline form” refers to crystals of a compound that have been washed and recrystallised to remove impurities.

The term “Amorphous” as used herein, relates to solid material which lacks a regular crystalline structure.

“Polymorphism” is the property of some molecular complexes to assume more than one crystalline or amorphous forms in the solid state. A single molecule like Aripiprazole may give rise to a variety of solids having distinct physical properties like solubility, X-ray diffraction pattern, IR spectrum and solid state ¹³C Nuclear Magnetic Resonance spectrum.

The differences in the physical properties of polymorphs result from the orientation and intermolecular interactions of adjacent molecules (complexes) in the bulk solid. Accordingly, polymorphs are considered as distinct solids sharing the same molecular formula.

The present invention relates to new forms of Aripiprazole, which is well distinguished from Aripiprazole as claimed in U.S. Pat. Nos. 4,734,416 and 5,006,528. These novel forms have been characterized by using DSC and X-ray powder diffraction.

Aripiprazole as claimed in U.S. Pat. No. 4,734,416 and U.S. Pat. No. 5,006,528 is crystallized from ethanol and has a distinct melting point of 139-139.5° C. In this specification we are considering this form as Form I.

The present invention discloses new forms of Aripiprazole obtained from the crude Aripiprazole using various solvents such as alcohols, ketones and esters. The new forms of Aripiprazole obtained by these processes have distinct melting points. The new forms also showed a marked difference from Aripiprazole as claimed in U.S. Pat. No. 4,734,416 and U.S. Pat. No. 5,006,528 with regards to DSC and XRD. The novel polymorphs of the present invention are:
polymorph having DSC endotherm in the range of 133-135° C. (described hereinafter as Form II)
polymorph having DSC endotherm in the range of 122-125° C. (described hereinafter as Form III)
polymorph having DSC endotherm in the range of 146-149° C. (described hereinafter as Form IV)

The present invention also describes methods for the preparation of polymorphic forms of Aripiprazole as well as their usage in medicine.

The polymorph having DSC endotherm in the range of 133-137° C. (Form II) can be prepared by contacting/dissolving crude Aripiprazole with suitable solvents like isopropanol, isopropyl acetate, methanol and the like or mixtures thereof and removing the solvent to obtain the desired-product.

The polymorph having DSC endotherm in the range of 122-124° C. (Form III) can be prepared by contacting/dissolving crude Aripiprazole with suitable solvents like isobutyl acetate, ethanol and the like or mixtures thereof and removing the solvent to obtain the desired product.

The polymorph having DSC endotherm in the range of 146-149° C. can be prepared by contacting/dissolving crude Aripiprazole with suitable solvents like acetone, f-butanol and the like or mixtures thereof and removing the solvent to obtain the desired product.

The polymorph having DSC endotherm in the range of 146-149° C. may also be obtained from a mixture of the other polymorphs by heating upto ca. 150° C. and subsequent cooling.

Any of the polymorphs can also be prepared by contacting/dissolving crude Aripiprazole with appropriate solvents, seeding that particular Form and removing the solvent to obtain the desired Form.

In a preferred embodiment, crude Aripiprazole used in these processes are prepared by following the process described in our Patent Application No. 793/MUM/2003.

The novel polymorphs of the present invention may easily be converted into their acid-addition salts by reacting with pharmaceutically acceptable acids. Examples of such acids include inorganic acids, such as hydrochloric acid, sulfuric acid, phosphoric acid, hydrobromic acid and the like; and organic acids such as oxalic acid, maleic acid, fumaric acid, malic acid, tartaric acid, citric acid, benzoic acid and the like.

The novel forms of Aripiprazole of the present invention can be used alone or in the form of suitable pharmaceutical compositions containing the novel forms together with the suitable pharmaceutically acceptable carriers.

The pharmaceutical compositions containing the novel forms of Aripiprazole of the present invention are prepared according to well known processes.

The dosage of the present central nervous controlling agents are suitably selected according to the usage, and may vary as per the requirement of the patient. The novel polymorphs of Aripiprazole described in the present invention are suitable as a central nervous controlling agent, preferably for the treatment of mental disorders like schizophrenia, Alzheimer's dementia, antipsychotic disorders and bipolar disorders

The present invention is illustrated by the following examples which should not be construed as limiting the scope of the invention.

### EXAMPLE 1

Aripiprazole (25 g) in 250 ml isopropyl acetate was stirred at reflux temperature for 30-90 mins and cooled. Crystallisation occurs during cooling process. The mixture was then brought to room temperature, filtered and washed with isopropyl acetate. The crystals were dried in an oven to constant weight to obtain Form II of Aripiprazole.

m.p.: 132-134° C.

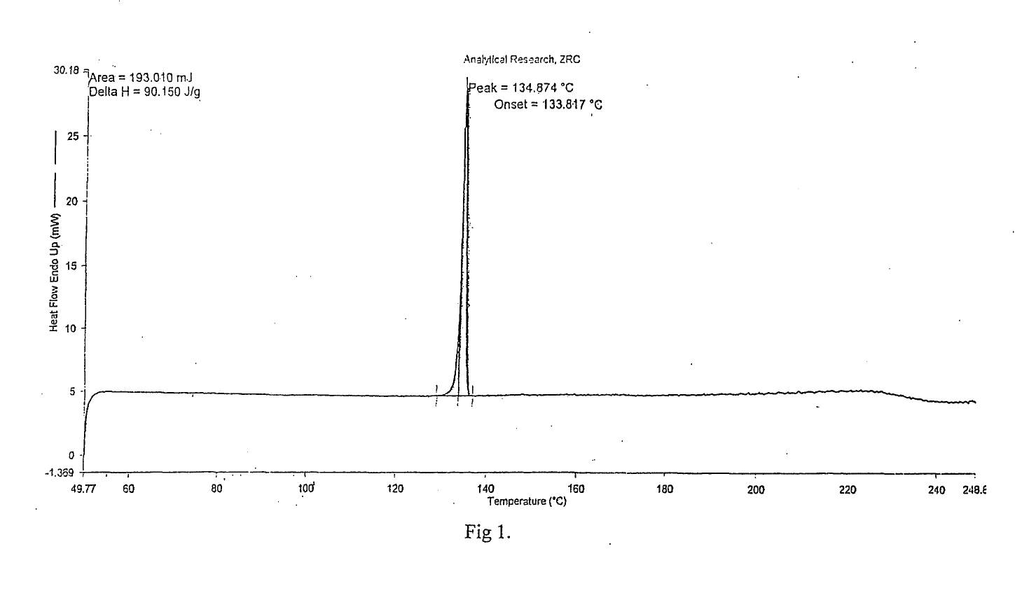

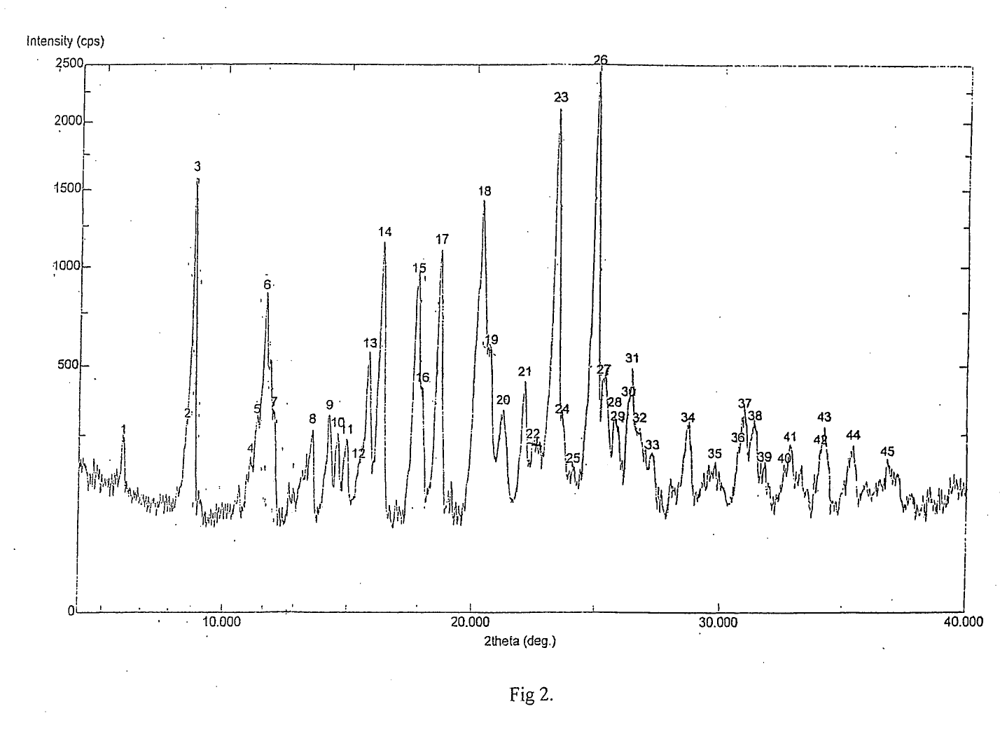

DSC and AD pattern similar to FIG. 1 and FIG. 2, respectively.

### EXAMPLE 2

The above procedure was followed using methanol as a solvent to obtain Form II of Aripiprazole.

m.p.: 132-134° C.

DSC and XRD pattern similar to FIG. 1 and FIG. 2, respectively.

### EXAMPLE 3

A mixture of 25 g crude Aripiprazole and 250 ml of isopropanol was stirred at reflux temperature to obtain a clear solution. The solution was cooled and seeding of Form II was added. Crystallisation occurs after some time. The solid was filtered and washed with 10 ml isopropanol. The product was dried in an oven to constant weight to get Form II of Aripiprazole.

m.p.: 132-134° C.

DSC and SD pattern similar to FIG. 1 and FIG. 2, respectively.

### EXAMPLE 4

A mixture of 25 g Aripiprazole and 250 ml isobutyl acetate was stirred at reflux temperature to obtain a clear solution is obtained. After stirring for 30-90 min the solution was cooled. Crystallisation occurs during the cooling process. The solids were filtered and washed with isobutyl acetate and dried in an oven to constant weight to obtain Form III of Aripiprazole.

m.p.: 122-124° C.

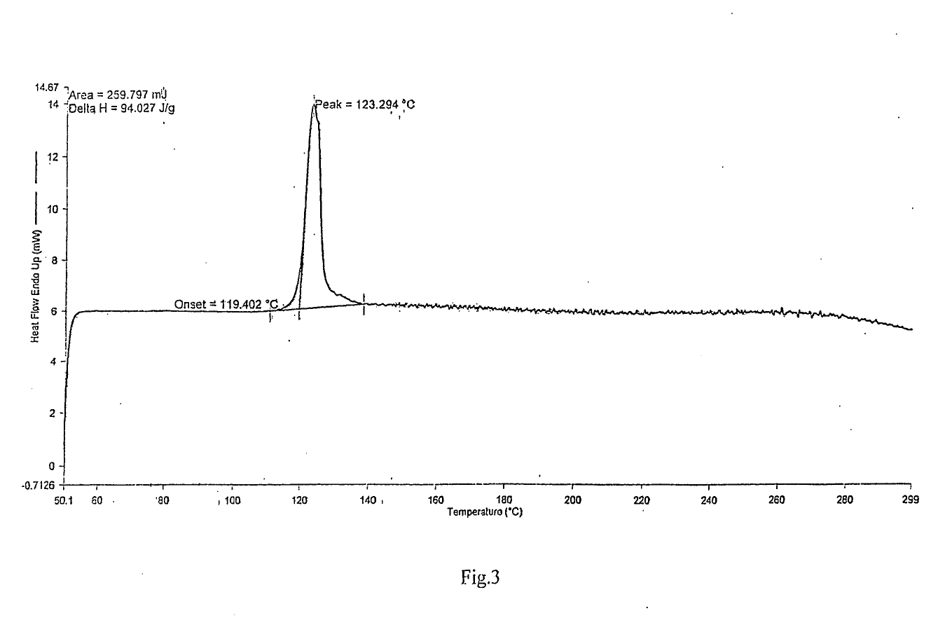

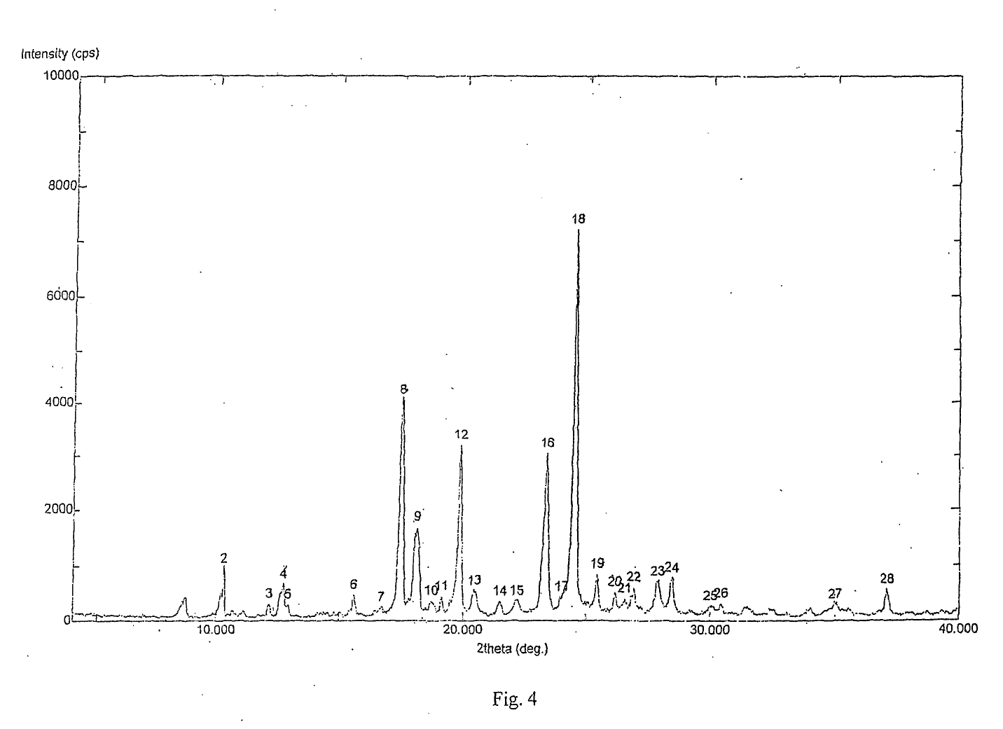

DSC and NRD pattern similar to FIG. 3 and FIG. 4, respectively.

We claim:

1. A novel polymorph of Aripiprazole characterized by data selected from the group comprising of DSC thermogram with an endothermic peak in the range of 133-137° C., X-ray diffraction pattern with peaks at about 5.820, 8.730, 11.640, 15.800, 16.310, 17.710, 18.610, 21.220, 22.090, 23.390, 24.950, 26.410, 30.970 and 34.190±0.2 degrees two-theta.

2. Novel polymorph of Aripiprazole as claimed in claim 1, with DSC pattern substantially as depicted in FIG. 1.

3. A novel polymorph of Aripiprazole as claimed in claim 1, characterized by X-ray diffraction pattern substantially as depicted in FIG. 2.

4. A novel polymorph of Aripiprazole characterized by data selected from the group comprising of DSC thermogram with an endothermic peak in the range of 122-124° C., X-ray diffraction pattern with peaks at about 8.730, 10.310, 12.170, 15.600, 16.700, 17.490, 19.30, 19.850, 20.440, 21.490, 22.180, 23.370, 24.510, 25.450, 26.940, 27.910, 28.470, 34.980, 37.080±0.2 degrees two-theta.

5. A novel polymorph of Aripiprazole as claimed in claim 4, with DSC pattern substantially as depicted in FIG. 3.

6. A novel polymorph of Aripiprazole as claimed in claim 4, characterized by X-ray diffraction pattern substantially as depicted in FIG. 4.

7. A novel polymorph of Aripiprazole characterized by data selected from the group comprising of DSC thermogram with an endothermic peak in the range of 146-149° C., X-ray diffraction pattern with peals at about 5.510, 10.250, 11.580, 12.760, 13.890, 15.540, 16.130, 17.240, 18.170, 18.540, 19.080, 20.740, 21.550, 22.240, 23.660, 24.740, 25.140, 25.740, 26.480, 27.010, 28.320, 29.450, 31.000, 32.600, 33.600, 35.590, 36.980, 38.840±0.2 degrees two-theta.

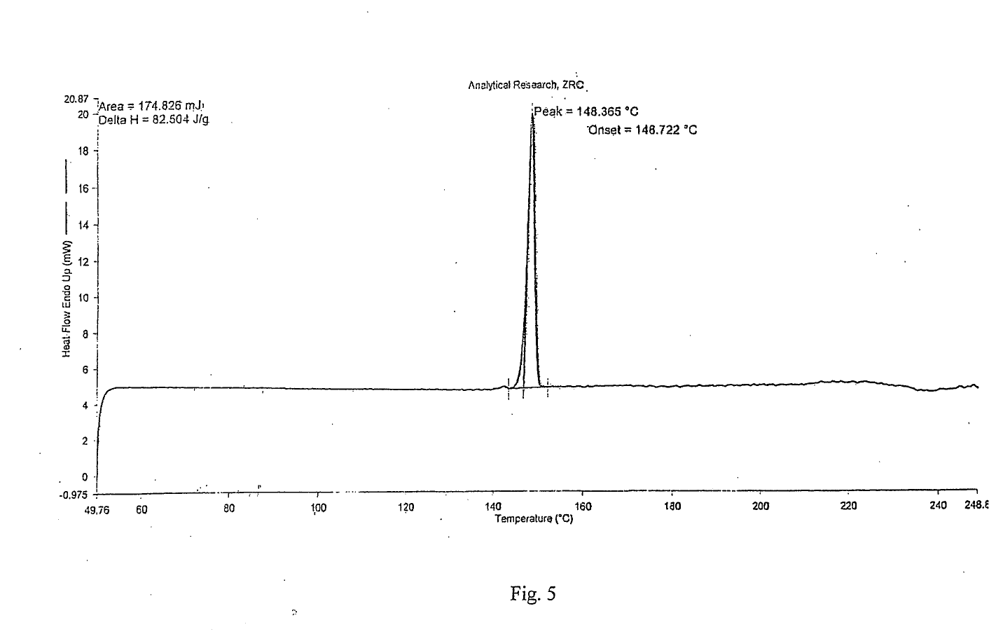

8. A novel polymorph of Aripiprazole as claimed in claim 7, with DSC pattern substantially as depicted in FIG. 5.

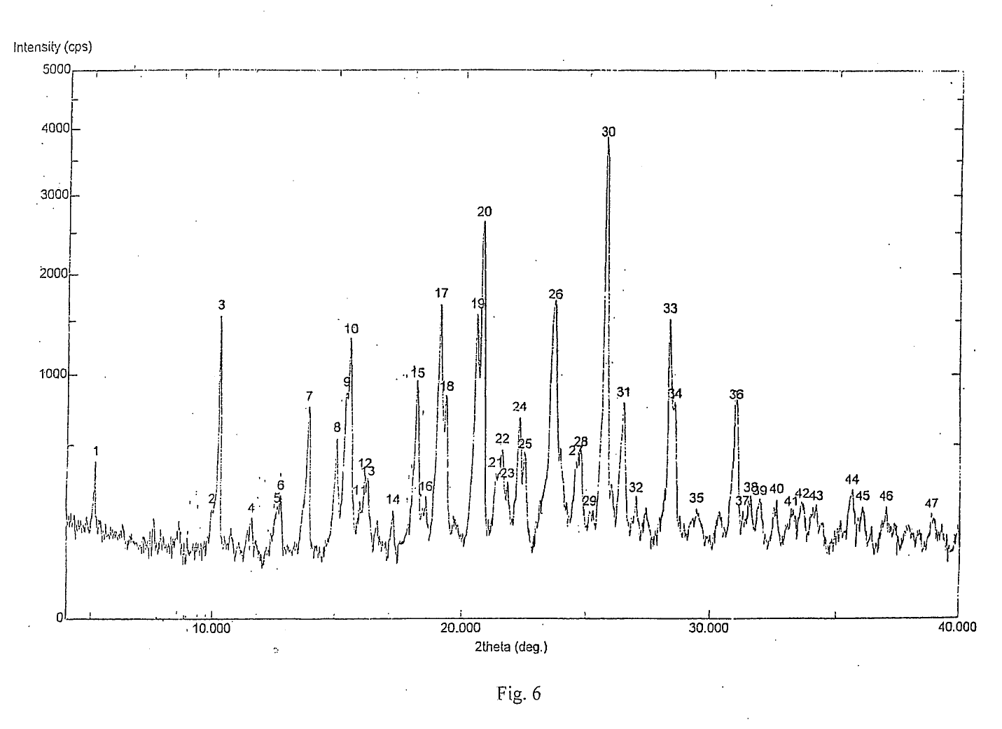

9. A novel polymorph of Aripiprazole as claimed in claim 7, characterized by X-ray diffraction pattern substantially as depicted in FIG. 6.

10. A process for the preparation of the novel polymorph as claimed in claims 1, 2, or 3 comprising,
c) contacting/dissolving crude Aripiprazole with suitable solvents selected from the group consisting of isopropanol, isopropyl acetate, methanol or mixtures thereof, at elevated temperature followed by cooling.
d) removing the solvent.

11. A process for the preparation of the polymorph as claimed in claims 4, 5 or 6 comprising,
c) contacting/dissolving crude Aripiprazole with suitable solvents selected from the group consisting of isobutyl acetate, ethanol or mixtures thereof, at elevated temperature followed by cooling.
d) removing the solvent.

12. A process for the preparation of the polymorph as claimed in claims 7, 8 or 9 comprising,
c) contacting/dissolving crude Aripiprazole with suitable solvents selected from the group consisting of acetone, t-butanol or mixtures thereof, at elevated temperature followed by cooling.
d) removing the solvent.

13. A process for the preparation of the polymorph as claimed in claim 7, 8 or 9 comprising, heating either pure polymorphs Form I-Form IV of Aripiprazole, or a mixture of two or more polymorphs of Aripiprazole upto ca. 150° C. and cooling.

14. A pharmaceutical composition comprising the novel polymorphs of Aripiprazole as claimed in any one of claims 1 to 6, consisting either a single polymorph or their mixtures in combination with pharmaceutically acceptable excipients.

15. A pharmaceutical dosage form comprising the pharmaceutical compositions containing the novel polymorphs of Aripiprazole as claimed in claim 14.

16. Use of the novel forms of Aripiprazole or pharmaceutical compositions containing them as claimed in any one of claims 1 to 8, 14 or 15 for preparing medicaments suitable for the treatment of conditions of the central nervous system such as schizophrenia.

17. Method of treatment of disorders of the central nervous system comprising administering to a person in need thereof, pharmaceutical compositions or pharmaceutically acceptable dosage forms containing the new forms of Aripiprazole any one of claims 1 to 8 and 14.
